(19) United States
(12) Patent Application Publication
(10) Pub. No.: US 2023/0049758 A1
(43) Pub. Date: Feb. 16, 2023

(54) ASSIGNMENT OF CLINICAL IMAGE STUDIES USING ONLINE LEARNING

(71) Applicant: INTERNATIONAL BUSINESS MACHINES CORPORATION, Armonk, NY (US)

(72) Inventors: Sun Young Park, San Diego, CA (US); Dustin Michael Sargent, San Diego, CA (US)

(21) Appl. No.: 17/402,040

(22) Filed: Aug. 13, 2021

(51) Int. Cl. G16H 30/40; G06N 20/00; G16H 30/20; G06K 9/62

(52) U.S. Cl. CPC G16H 30/40; G06N 20/00; G16H 30/20; G06K 9/6263; G06K 9/6253

(57) ABSTRACT

Methods and systems for training a model using machine learning for automatically distributing medical imaging studies to radiologists. One method includes receiving one or more medical images included in a medical study, each of the one or more medical images including image metadata defining characteristics of the corresponding medical image. The method further includes receiving radiologist metadata for each one of the plurality of radiologists, generating a state representation of the image metadata and the radiologist metadata, and providing the state representation to the model. The method further includes assigning, with the model, at least one of the one or more medical images to one of the plurality of radiologists, calculating feedback based on a change in the state representation after the at least one of the one or more medical images is assigned to one of the plurality of radiologists, and adjusting the model based on the feedback.

### FIELD

Embodiments described herein relate to systems and methods for automatically assigning clinical studies. Some systems and methods use online learning, such as reinforcement learning, to build a model for assigning images, wherein the model handles non-linear relationships between assignment goals and parameters, such as, for example, radiologist preference and fairness.

### SUMMARY

Image studies may be assigned to radiologists for analysis via a worklist. A worklist can include image studies that need to be analyzed by a specific radiologist or team of radiologists (e.g., a group of radiologists within a given lab, department, hospital, and the like). Image studies can be assigned manually, where a radiologist can select an image study from a pool of available images studies. Manual selection, however, may allow “cherry-picking” where a radiologist may select only his or her favorite or preferred image studies (e.g., simple image studies may be picked over more complex image studies). Accordingly, resentment may build among the remainder of a radiologist team, as a radiologist may be left to analyze his or her “least-favorite” image studies. Additionally, undesired image studies may await review for an extend period as time, as they are intentionally avoided. Similarly, in systems where thousands of studies may be received for per hour for assignment to a radiologist, image studies may sit for extended periods until a study is manually selected by a radiologist. Furthermore, when a radiologist can manually select an image study, studies may not necessarily be properly matched with a radiologist with the right specialty and expertise.

Image studies can also be assigned using rule-based systems. These rule-based systems, however, require extensive initial configuration or definition and cannot adapt or learn over time. Additionally, simple logic rules cannot balance for the relationship between study complexity, study priority, radiologist preference, and radiologist capacity. For example, a rule-based system may be configured to assign a newly received image study to the radiologist who currently has the lowest number of studies assigned. This type of rule can effectively punish hard-working radiologists and, like manual assignment, can create inefficient or unfair workload balances and may also fail to account for the specialty or expertise of a particular radiologist.

Accordingly, embodiments described herein provide methods and system for training and implementing a model for worklist assignment. The methods and systems can use online learning, wherein data obtained over a period of time is used to update the model for future data (e.g., as compared to batch learning techniques). In particular, embodiments described herein can use a reinforcement learning model. Reinforcement learning is an area of machine learning directed to learning an action to take in an environment to maximize a reward. Unlike supervised learning, reinforcement learning does not require labeled input/output pairs, which is difficult if not impossible to acquire for an image study assignment environment where there is generally not a true or correct answer (i.e., assignment) given the many parameters and often conflicting goals associated with study assignment.

Reinforcement learning can be modeled as a Markov decision process (MDP) where S represents a set of environment states, A represents a set of actions, P represents a probability of transition from one state to another state via one of the actions, and R represents a reward associated with a transition from one state to another state via one of the actions. Using this process, the model learns an optimal (or nearly optimal) policy that maximizes the reward. In particular, at each time t, the model receives the current state and choses an action from a set of available actions. The environment moves to a new state based on the chosen action, and a reward associated with the transition to the new state is determined. The determined reward is used as feedback for the model, wherein the model uses the feedback to learn a policy that maximizes expected cumulative reward.

For example, a reinforcement learning model receives a plurality of image studies to be assigned and radiologist information (e.g., metadata) describing each available radiologist. Each image study also has its own metadata describing the image study, such as the image modality and study complexity. The model uses the metadata of each image study and the metadata describing each radiologist to assign each image study to a radiologist. Following assignment, the model receives feedback. Among other types of feedback, the feedback may indicate whether or not the radiologist is pleased with the assignment or whether the assignment was appropriate (e.g., whether the radiologist rejected the assignment). The feedback may also include timing information, such as the radiologist's reading time associated with the assigned image study or how long it took, from assignment, for the radiologist to complete his or her review of the image study. The model uses the feedback to continue to optimize the assignment policy implemented via the model.

In addition to removing the time required for manual workload distribution, using a machine learning model as described herein to assign image studies to radiologists provides a balance between radiologist preference and a fairness in assignment. To achieve this balance, the machine learning model targets the fairest distribution of workload using a plurality of instructions or conditions (e.g., a fairness criteria). Additionally, the machine learning model may constantly request and receive updated metadata for each radiologist to maintain an accurate representation of preferences and workload. Furthermore, as compared to simple rules-based assignment system, the machine learning model as described herein requires less initial configuration and can automatically adjust overtime to changing preferences or other parameters.

Accordingly, embodiments described herein use online learning models, such as reinforcement-based learning models, to automatically assign medical image studies to radiologists. For example, one embodiment provides a computer-implemented method of training a model using machine learning for automatically distributing medical imaging studies to radiologists. The method includes receiving one or more medical images included in a medical study, each of the one or more medical images including image metadata defining characteristics of the corresponding medical image. The method includes receiving radiologist metadata for each one of the plurality of radiologists, generating a state representation of the image metadata and the radiologist metadata, and providing the state representation to the model. The method includes assigning, with the model, at least one of the one or more medical images to one of the plurality of radiologists, calculating feedback based on a change in the state representation after the at least one of the one or more medical images is assigned to one of the plurality of radiologists, and adjusting the model based on the feedback.

Another embodiment provides a system for training a model using machine learning for automatically distributing medical imaging studies to radiologists. The system includes an electronic processor. The electronic processor is configured to receive one or more medical images included in a medical study, each of the one or more medical images including image metadata defining characteristics of the corresponding medical image. The electronic processor is further configured to receive radiologist metadata for each one of the plurality of radiologists, generate a state representation of the image metadata and the radiologist metadata, and provide the state representation to the model. The electronic processor is further configured to assign, with the model, at least one of the one or more medical images to one of the plurality of radiologists, calculate feedback based on a change in the state representation after the at least one of the one or more medical images is assigned to one of the plurality of radiologists, and adjust the model based on the feedback.

A further embodiment provides non-transitory computer-readable medium storing instructions that, when executed by an electronic processor, perform a set of functions. The set of functions includes receiving one or more medical images included in a medical study, each of the one or more medical images including image metadata defining characteristics of the corresponding medical image. The set of functions includes receiving radiologist metadata for each one of the plurality of radiologists, generating a state representation of the image metadata and the radiologist metadata, and providing the state representation to the model. The set of functions includes assigning, with the model, at least one of the one or more medical images to one of the plurality of radiologists, calculating feedback based on a change in the state representation after the at least one of the one or more medical images is assigned to one of the plurality of radiologists, and adjusting the model based on the feedback.

Other aspects of the embodiments will become apparent by consideration of the detailed description and accompanying drawings.

### BRIEF DESCRIPTION OF THE DRAWINGS

FIG. 1 schematically illustrates an image study assignment system according to some embodiments.

FIG. 2 schematically illustrates assignment of image studies to individual radiologist worklists according to some embodiments.

FIG. 3 illustrates example metadata received by an online learning model according to some embodiments.

FIG. 4 illustrates a state representation format of metadata received by the online learning model of FIG. 3.

FIG. 5 schematically illustrates a reward system for a reinforcement learning model according to some embodiments.

FIG. 6 is a flowchart illustrating a method performed by the image study assignment system of FIG. 1.

### DETAILED DESCRIPTION

Before any embodiments are explained in detail, it is to be understood that the embodiments are not limited in their application to the details of construction and the arrangement of components set forth in the following description or illustrated in the following drawings. Other embodiments are capable of being practiced or of being carried out in various ways.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and may include electrical connections or coupling, whether direct or indirect. Also, electronic communications and notifications may be performed using any known means including direct connections, wireless connections, etc.

A plurality of hardware and software-based devices, as well as a plurality of different structural components may be utilized to implement the embodiments. In addition, embodiments may include hardware, software, and electronic components or modules that, for purposes of discussion, may be illustrated and described as if the majority of the components were implemented solely in hardware. However, one of ordinary skill in the art, and based on a reading of this detailed description, would recognized that, in at least one embodiment, the electronic-based aspects of the embodiments may be implemented in software (e.g., stored on non-transitory computer-readable medium) executable by one or more processors. As such, it should be noted that a plurality of hardware and software-based devices, as well as a plurality of different structural components, may be utilized to implement the embodiments. For example, “mobile device,” “computing device,” and “server” as described in the specification may include one or more electronic processors, one or more memory modules including non-transitory computer-readable medium, one or more input/output interfaces, and various connections (e.g., a system bus) connecting the components.

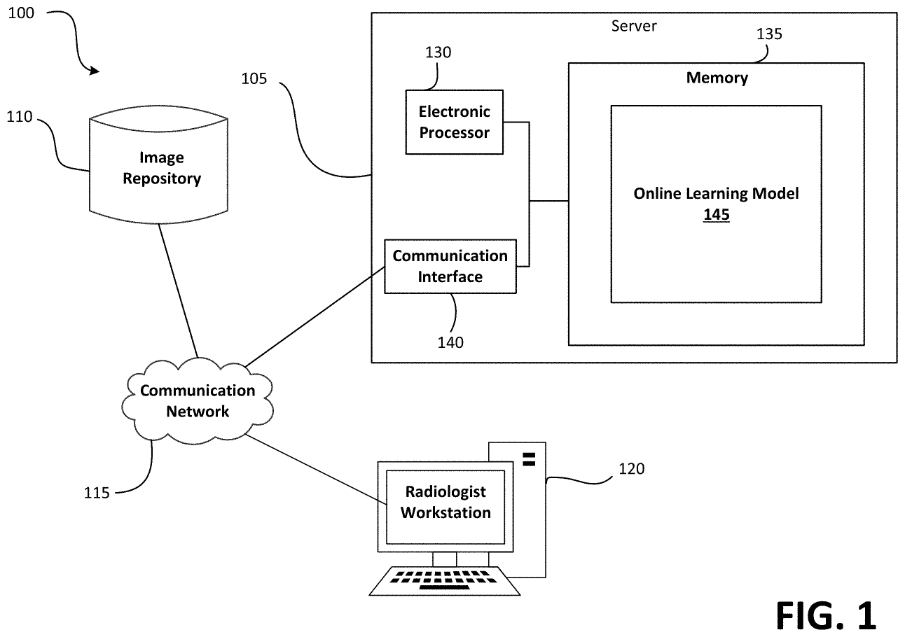

As described above, embodiments provided herein provide methods and systems for an online learning model for medical image study worklist assignment. FIG. 1 illustrates an image study assignment system 100 according to some embodiments. As illustrated in FIG. 1, the system 100 includes a server 105, an image repository 110, and a workstation 120. The server 105, the image repository 110, and the workstation 120 communicate over one or more wired or wireless communication networks 115. Portions of the wireless communication networks 115 may be implemented using a wide area network, such as the Internet, a local area network, such as a Bluetooth™ network or Wi-Fi, and combinations or derivatives thereof. It should be understood that the system 100 may include more or fewer servers and the single server 105 illustrated in FIG. 1 is purely for illustrative purposes. For example, in some embodiments, the functionality described herein is performed via a plurality of servers in a distributed or cloud-computing environment. Also, in some embodiments, the server 105 may communicate with multiple image repositories. Additionally, it should be understood that the system 100 may include more workstations and the single workstation 120 illustrated in FIG. 1 is purely for illustrative purposes. For example, in some embodiments, the system 100 includes a plurality of workstations 120, each workstation associated with a radiologist. Also, in some embodiments, the components illustrated in system 100 may communicate through one or more intermediary devices (not shown).

The image repository 110 stores two-dimensional images, three-dimensional image volumes, or both in the image repository 110. The image repository 110 may be, for example, a picture archiving and communication system (PACS), a cloud storage environment, or the like. The images stored in the image repository 110 are generated by an imaging modality (not shown), such as an X-ray computed tomography (CT) scanner, a magnetic resonance imaging (Mill) scanner, or the like. In some embodiments, the image repository 110 may also be included as part of an imaging modality. The images stored in the image repository 110 may be grouped into image studies. An image study may comprise one or more images related to, for example, a specific patient, a specific anatomical location, or the like. In some embodiments, images within an image study were generated by the same image modality.

As illustrated in FIG. 1, the server 105 includes an electronic processor 130, a memory 135, and a communication interface 140. The electronic processor 130, the memory 135, and the communication interface 140 communicate wirelessly, over wired communication channels or buses, or a combination thereof. The server 105 may include additional components than those illustrated in FIG. 1 in various configurations. For example, in some embodiments, the server 105 includes multiple electronic processors, multiple memory modules, multiple communication interfaces, or a combination thereof. Also, it should be understood that the functionality described herein as being performed by the server 105 may be performed in a distributed nature by a plurality of computers located in various geographic locations. For example, the functionality described herein as being performed by the server 105 may be performed by a plurality of computers included in a cloud computing environment.

The electronic processor 130 may be, for example, a microprocessor, an application-specific integrated circuit (ASIC), and the like. The electronic processor 130 is generally configured to execute software instructions to perform a set of functions, including the functions described herein. The memory 135 includes a non-transitory computer-readable medium and stores data, including instructions executable by the electronic processor 130. The communication interface 140 may be, for example, a wired or wireless transceiver or port, for communication over the communication network 115 and, optionally, one or more additional communication networks or connections.

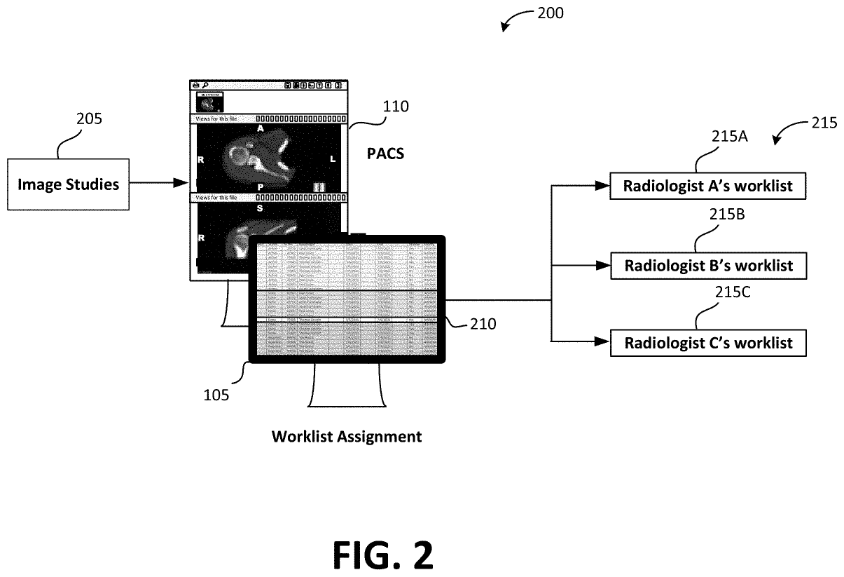

As illustrated in FIG. 1, the memory 135 of the server 105 includes an online learning model 145, which may be part of a study assignment engine executed via the server 105. The online learning model 145 may be, for example, a reinforcement learning model. Additionally, the memory 135 may store a worklist assignment table that identifies which image studies are assigned to each radiologist working within the system 100. As image studies are provided to the image repository 110, the server 105 uses the online learning model 145 to assign the image studies to radiologists (i.e., radiologist worklists) within the system 100. For example, FIG. 2 illustrates a workflow 200 for assigning image studies to radiologists. As illustrated in FIG. 2, a plurality of image studies 205 are stored to the image repository 110 (for example, a PACS), and server 105 stores (or has access to) a worklist assignment table 210 that includes, among other things, an identifier for each image study of the plurality of image studies 205 and a status of each image study. The server 105 also stores (or has access to) a plurality of radiologist worklists 215. As described in further detail below, the server 105 uses the online learning model 145 to assign each image study of the plurality of image studies 205 to one of the radiologist worklists 215 (such as, for example, the radiologist A worklist 215A, the radiologist B worklist 215B, or the radiologist C worklist 215C).

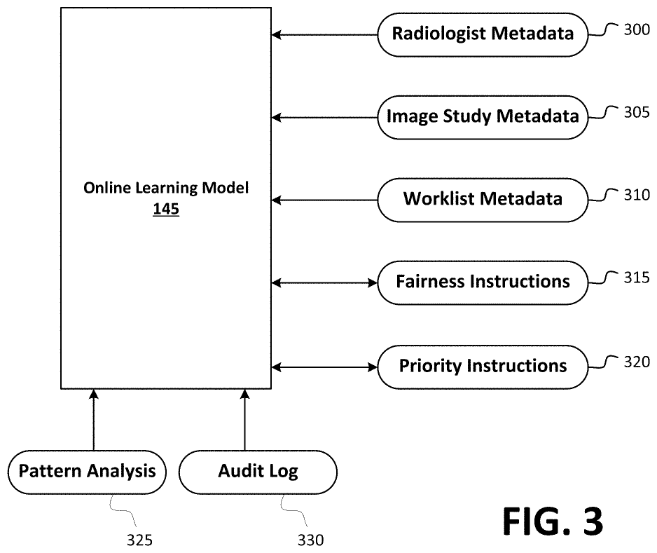

As shown in FIG. 3, the online learning model 145 assigns image studies to radiologists based on several factors. For example, as shown in FIG. 3, the online learning model 145 receives a plurality of input parameters, such as radiologist metadata 300, image study metadata 305, worklist metadata 310, fairness instructions 315, priority instructions 320, and pattern analysis 325. The image study metadata 305 is associated with an incoming image study. The image study metadata 305 may include, for example, an arrival or retrieval time of the image study, a due date or time of the image study, a modality used to generated images include in the image study, an imaging procedure associated with the image study (e.g., contrast versus no contrast), one or more anatomical structures represented within images of the image study, a complexity of the image study, a description of the image study, or a combination thereof. Additionally, each medical image included in the image study may have its own associated metadata in addition to metadata defining the image study as a whole. Accordingly, the image study metadata 305 may include, for example, an arrival or retrieval time of each medical image, a due date or time of each medical image, a modality used to generate each medical image, an imaging procedure associated with each medical image (e.g., contrast versus no contrast), one or more anatomical structures represented within each medical image, a complexity of each medical image, a description of each medical image, or a combination thereof. In some embodiments, the image study metadata 305 also includes patient metadata, such as a gender, age, medical condition, ethnicity, geographic location, or the like.

The worklist metadata 310 includes information describing the worklist assignment table 210, such as available radiologists (e.g., an identifier of each available radiologist), a status of each radiologist (for example, a number of image studies currently assigned to each radiologist), a status of the image studies themselves (for example, whether the radiologist has begun to analyze the image study and whether the radiologist has completed their analysis of the image study), and, optionally, any image studies each radiologist has rejected. The radiologist metadata 300 may also include additional information regarding a radiologist, such as a radiologist's personal preferences (such as a favorite type of image modality), a radiologist's specialty, qualifications, or expertise, an average capacity-per-hour of each radiologist, an average reading time of each radiologist, or the like. In some embodiments, the radiologist metadata 300 is gathered from a profile of each radiologist, which may be manually configured by the radiologist or a system administrator. In other embodiments, the server 105 may maintain information or statistics of each radiologists (e.g., within the memory 135), which may be used to generate a portion of the radiologist metadata 300. For example, the server 105 may track or receive information regarding reading time for a particular radiologist, which may be used to provide personized reading time analysis and associated image study assignment. Similarly, the server 105 may track study rejection by radiologists, which may be used to automatically learn preferences of particular radiologists.

While FIG. 3 illustrates the radiologist metadata 300, the image study metadata 305, and the worklist metadata 310 as separate inputs, in some embodiments, the server 105 may receive the inputs in various combinations and from various sources. Accordingly, the radiologist metadata 300, the image study metadata 305, and the worklist metadata 310 are shown as separate inputs in FIG. 3 for illustrative purposes.

In some embodiments, the online learning model also receives fairness instructions 315 and priority instructions 320. The fairness instructions 315 include one or more rules describing balancing and weighting of the inputs to achieve a fair work distribution. In other words, the fairness instructions 315 define fairness criteria for image study assignment. These rules may include, for example, assigning the received image study to the radiologist with the least studies currently assigned, assigning the received image study to the radiologist with the fastest expected reading time for the study, and the like. The online learning model 145 uses the fairness instructions 315 as part of determining which radiologist to assign an incoming image study to. Accordingly, while the fairness instructions 315 may be definite rules, the online learning model 145 converts the fairness instructions 315 into suggestions which are accounted for. In some embodiments, the online learning model orders the plurality of available radiologists in a list sorted from best choice to worst choice according to each rule included in the fairness instructions 315. The online learning model 145 then uses each list (i.e., the list generated for each rule) as an input to determine which radiologist to assign the incoming image study to.

The online learning model may also receive priority instructions 320. The priority instructions 320 defines exceptions to the fairness instructions 315. For example, an urgent image study may be received. The online learning model 145 identifies the image study as urgent, and uses rules defined by the priority instructions 320. The online learning model 145 may identify only a single radiologist that can meet the deadline for the urgent study. While the radiologist may already have assigned image studies, the online learning model 145 may ignore the current workload to still assign the image study to that radiologist. Accordingly, the priority instructions 320 may include assignment to a radiologist with the greatest capacity, assignment to a radiologist with the fastest reading time for the required image study type, assignment to a radiologist with a history of meeting deadlines, or a combination thereof. In some embodiments, the fairness instructions 315 and the priority instructions 320 are both stored by and implemented by the online learning model 145.

The online learning model 145 may also receive a pattern analysis 325. The pattern analysis 325 includes information detailing historical patterns of each radiologist. For example, the pattern analysis 325 may describe types of image studies that are typically rejected by each radiologist and an average amount of time certain types of image studies (such as specific image modalities) take each radiologist to analyze. Such pattern information of each radiologist may include, for example, an average and standard deviation of: reading time for each image study type, an RVU rate for each image study type, an RVU rate per hour, an RVU rate per day, a rate of meeting deadlines, a rejection rate for each image study type, a percentage of each study type assigned to the given radiologist, and the like. In some embodiments, the online learning model 145 also receives information from an audit log 330 containing a record of activity within the image repository 110. For example, the audit log information may include records of which radiologists were assigned which image studies, a date and time at which image studies were initially assigned, a date and time at which analysis of each image study was completed, whether studies were rejected or manually reassigned, and the like.

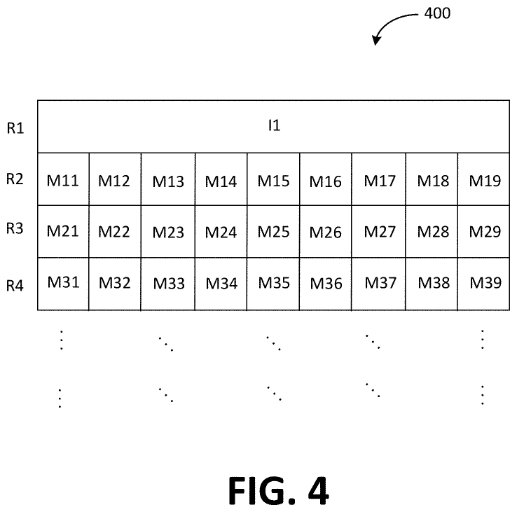

The online learning model 145 may receive the image study metadata 305 and the radiologist metadata 300 in a predetermined state representation. Alternatively, the online learning model 145 may generate a state representation based on the received image study metadata 305 and radiologist metadata 300. For example, FIG. 4 illustrates a state representation 400 including the image study metadata 305 and radiologist metadata 300 as horizontal vectors. In the illustrated example of FIG. 4, the state representation 400 includes a first row (or first vector) R1, a second row R2, a third row R3, and a fourth row R4. The first row R1 includes image study metadata I1. The image study metadata I1 is associated with an incoming image study to be assigned. The subsequent rows R2-R4 each include metadata associated with a radiologist available to be assigned the incoming image study. The metadata associated with each radiologist may include, for example, currently assigned image studies, preferences of the radiologist, and radiologist analysis patterns (such as, for example, reading statistics, previous rejections, and current workload). Accordingly, each row R2-R4 may provide a detailed status of each available radiologist.

Each column within a row of the state representation 400 describes a specific metadata. For example, each column of metadata may describe, among other things, the specialty of each corresponding radiologist (defined by each row), the capacity of each corresponding radiologist, the capacity to receive a high priority (or urgent) image study of each corresponding radiologist, preference statistics of each corresponding radiologist, an estimated time it would take each corresponding radiologist to read the incoming image study, a current relative value unit (RVU) of each corresponding radiologist, a complexity of each corresponding radiologists' current workload, and a reading time of each corresponding radiologists' current workload.

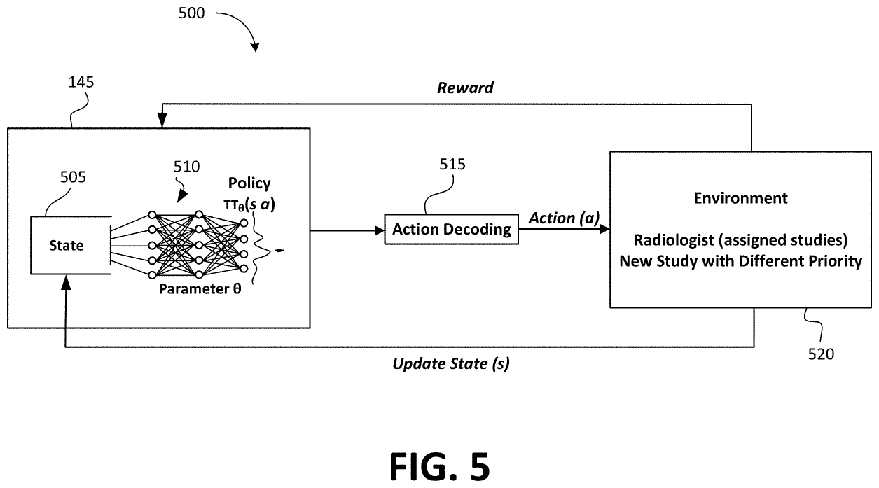

As the online learning model 145 assigns incoming image studies to radiologists within the system 100, the online learning model 145 receives feedback. The online learning model 145 updates its own parameters and decision-making processes based on the received feedback. For example, FIG. 5 schematically illustrates a reward system 500 for the online learning model when the online learning model includes a reinforcement learning model. As illustrated in FIG. 5, in this implementation, the online learning model 145 maintains a current state 505 and a neural network 510 (for example, a deep neural network [DNN]). The current state 505 represents metadata received by the online learning model 145, such as, for example, radiologist metadata 300, image study metadata 305, and worklist metadata 310. In other words, the state represents metadata regarding an incoming image study needing assignment and a state or status of each radiologist. The neural network 510 receives the state 505 and provides an output or action (e.g., a selected radiologist to receive the incoming image study) based on the state 505.

The output of the neural network 510 is provided to an environment block 520. In some embodiments, the output is provided to an action decoding block 515 prior to being provided to an environment block 520. The environment block 520 represents an updated state of the system 100 following assignment of the incoming image study. For example, as incoming studies are assigned to radiologists, their capacity for new projects changes. Accordingly, the environment block 520 reflects a changing (e.g., updated) representation of the state 505 that is periodically updated, such as each time an assignment is made, each time a new image study is received needing assignment, or the like. For example, the environment block 520 includes an updated worklist 210. As one example, radiologists may reject assigned image studies. The environment block 520 includes these rejections and may provide a rejected image study to the online learning model to be reassigned. As new image studies are provided to the online learning model 145, each new image study has its own unique metadata included in the environment block 520. The environment block 520 then provides the updates to state 505.

In some embodiments, in addition to the updated worklist 210, a radiologist may revise their preferences, may move to a new team, and/or may be added to or removed from the system 100. Accordingly, server 105 may periodically request updated radiologist metadata from each radiologist workstation 120. This allows the neural network 145 to use the most current metadata regarding each radiologist within the system 100.

Following assignment of an incoming image study, the online learning model 145 may receive feedback based on the assignment. For example, the radiologist assigned the image study may indicate whether they were pleased with the assignment. This feedback may be a rejection or acceptance of the image study, may be indicated based on a prompt provided to the radiologist, or the like. Furthermore, in some embodiments, this radiologist-based feedback is inferred based on reading completion time. For example, if it takes the radiologist a long time to select an assigned image study from their worklist, this could indicate that the radiologist was not happy with their assignment or the assignment failed to take into account the radiologists existing workload. Similarly, if it takes the radiologist a long time to complete the review of an assigned image study, this could indicate that the radiologist was not happy with their assignment or that the assigned image study did not match the radiologist's specialty or preference. In some embodiments, feedback is calculated as a reward value. For example, each radiologist has a specialty included in the radiologist metadata 300. Each incoming image study has a body part identifier included in the image study metadata 305. If the specialty of the radiologist assigned the image study does not align with the body part identifier (e.g., the radiologist is not qualified to analyze the assigned image study), a negative reward is provided to the online learning model 145. Alternatively, if the specialty of the radiologist assigned the image study does align with the body part identifier (e.g., the radiologist is qualified to analyze the assigned image study), a positive reward is provided to the online learning model 145.

In another example, an incoming image study may be labelled as “urgent.” When an incoming image study is urgent, the online learning model 145 may select a specific priority instruction 320 based on the state 505. In this situation, the online learning model 145 may more heavily weigh the reading speed of each radiologist to handle such an image study. Accordingly, if the online learning model 145 provides an urgent incoming image study to a radiologist with a slow reading speed (as determined by radiologist metadata 300), a negative reward may be provided to the online learning model 145. Alternatively, if the online learning model 145 provides the incoming image study to a radiologist with a fast reading speed, a positive reward may be provided to the online learning model 145.

In another example, each radiologist has a preference included in the radiologist metadata 300. Each incoming image study has a modality included in the image study metadata 305. If the preference of the radiologist assigned the image study does not align with the modality (e.g., the radiologist dislikes the incoming modality), a negative reward is provided to the online learning model 145. Alternatively, if the preference of the radiologist assigned the image study does align with the modality, a positive reward is provided to the online learning model 145.

Rewards may also be calculated by observing changes between the state 505 and the environment 520. For example, rewards may be calculated based on changes in workload of the radiologists. If each radiologist has a similar workload, the calculated reward may be positive. However, if a large variance in workload grows between the radiologists, the calculated reward may be negative. In some embodiments, the value of rewards also varies based on the assignment of image studies. For example, a high value reward may be given when an assignment creates little change in the state 505. Additionally, a high reward may be given when an image study is assigned to the best possible candidate (such as who has the lowest current workload). A low value reward may be given when the assignment results in a change in the state 505 and the variance between radiologists is still considered fair.

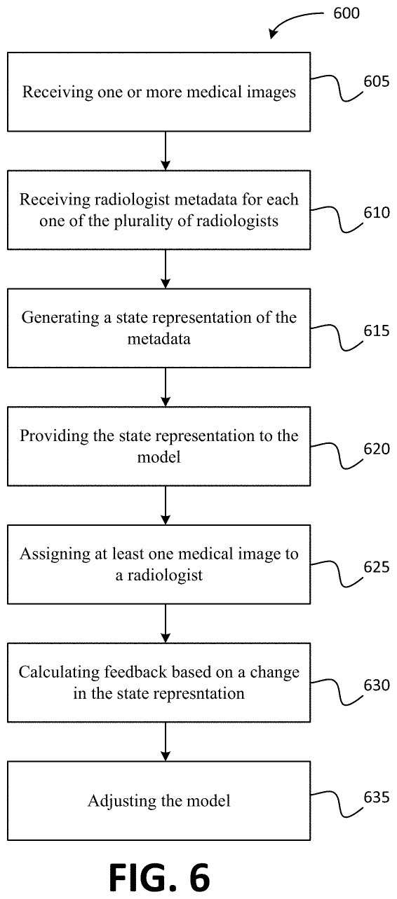

Accordingly, the electronic processor 130, with the online learning model 145, uses information related to the radiologist and information related to the image study itself to fairly distribute incoming image studies. FIG. 6 is a flowchart illustrating a method 600 for automatically distributing incoming medical image studies to radiologists. The method 600 may be performed by the server 105 (i.e., the electronic processor 130 implementing the online learning model 145). The method 600 includes receiving one or more medical images, such as a set of images included in an image study (at block 605). For example, an image study may be uploaded to the image repository 110 and the server 105 may receive metadata regarding the image study (e.g., through a push or pull configuration with the image repository). As noted above, in some embodiments, the image repository 110 includes a PACS, and the PACS can be configured to execute the study assignment engine (including the online learning model 145) as described herein.

The method 600 includes receiving the radiologist information for each one of the plurality of radiologists (at block 610). For example, each radiologist workstation 120 transmits its associated radiologist metadata 300 to the server 105. In some embodiments, the method 600 includes requesting radiologist information associated with a plurality of radiologists. For example, the server 105 transmits a request for radiologist metadata 300 to each radiologist workstation 120 included in the system 100. In some embodiments, rather than requesting radiologist information, each radiologist workstation 120 transmits its associated radiologist metadata 300 at predetermined time intervals. Additionally, the electronic processor 130 may obtain the worklist metadata 310 from the worklist 210 or from another data source storing information regarding radiologists.

The method 600 includes generating a state representation of the metadata (at block 615). For example, the electronic processor 130 generates the state representation 400 using the radiologist metadata 300 and the image study metadata 305. In some embodiments, the electronic processor 130 receives the worklist metadata 310 and includes the worklist metadata 310 in the state representation 400. The method 600 includes providing the state representation to the online learning model 145 (at block 620). For example, the state representation 400 may be provided to the neural network 510 as state 505 (see FIG. 5).

The method 600 includes assigning the image study to one of the plurality of radiologists (at block 625). For example, the online learning model 145 analyzes the received state representation 400 and provides an action (i.e., an assignment to one of the radiologists) as an output. In some embodiments, the entire image study is assigned to the selected radiologist. However, in other embodiments, only a portion of the image study may be assigned to the selected radiologist. For example, rather than assigning the entire image study to one radiologist, the online learning model 145 may assign portions of the image study to several radiologists.

The method 600 includes calculating feedback based on a change in the state representation (at block 630). For example, in response to assigning the image study to a particular radiologist, the state representation 400 is updated to reflect changes in the radiologist metadata 310. As previously described, a reward value is calculated based on these changes. In some embodiments, a radiologist also provides feedback indicating their satisfaction with the assigned image study. The reward value, the radiologist feedback, or both is provided to the online learning model 145 as feedback. In some embodiments, the radiologist feedback is used to calculate the reward value. As illustrated in FIG. 6, the method 600 also includes adjusting the online learning model based on the feedback (at block 635). For example, in some embodiments, the neural network 510 included in the online learning model 145 is updated based on the feedback.

Various features and advantages of the embodiments are set forth in the following claims.

What is claimed is:

1. A computer-implemented method of training a model using machine learning for automatically distributing medical imaging studies to radiologists, the method comprising:
receiving one or more medical images included in a medical study, each of the one or more medical images including image metadata defining characteristics of the corresponding medical image,
receiving radiologist metadata for each one of the plurality of radiologists,
generating a state representation of the image metadata and the radiologist metadata,
providing the state representation to the model,
assigning, with the model, at least one of the one or more medical images to one of the plurality of radiologists,
calculating feedback based on a change in the state representation after the at least one of the one or more medical images is assigned to one of the plurality of radiologists, and
adjusting the model based on the feedback.

2. The method of claim 1, wherein the image metadata of each of the one or more medical images includes at least one of an arrival time of the medical image, a due time of the medical image, a modality of the medical image, a procedure of the medial image, a body part of the medical image, and a description of the medical image.

3. The method of claim 1, wherein the radiologist metadata includes at least one of a specialty of each of the plurality of radiologists, a work list of each of the plurality of radiologists, an availability of each of the plurality of radiologists, a preference of each of the plurality of radiologists, and a processing rate of each of the plurality of radiologists.

4. The method of claim 1, wherein the state representation is a 2D table comprising:
a first row including the image metadata, and
subsequent rows including the radiologist metadata, wherein each row includes radiologist metadata regarding an individual radiologist.

5. The method of claim 1, further including updating the state representation with current radiologist metadata at predetermined time intervals.

6. The method of claim 1, wherein the feedback includes a rejection of the assigned one or more medical images.

7. The method of claim 1, further comprising providing a fairness-criteria to the model, the fairness-criteria including a plurality of conditions associated with the assignment of the one or more medical images.

8. The method of claim 7, further comprising calculating the feedback based on the fairness-criteria.

9. The method of claim 7, further comprising:
selecting one of the plurality of conditions,
ordering the plurality of radiologists in a list based on the selected one of the plurality of conditions, and
assigning, with the model, at least one of the one or more medical images to one of the plurality of radiologists based on the list.

10. The method of claim 1, further comprising:
calculating a variance in workload of each of the plurality of radiologists, and
calculating the feedback based on the variance.

11. A system for training a model using machine learning for automatically distributing medical imaging studies to radiologists, the system comprising:
an electronic processor configured to:
receive one or more medical images included in a medical study, each of the one or more medical images including image metadata defining characteristics of the corresponding medical image,
receive radiologist metadata for each one of the plurality of radiologists,
generate a state representation of the image metadata and the radiologist metadata,
provide the state representation to the model,
assign, with the model, at least one of the one or more medical images to one of the plurality of radiologists,
calculate feedback based on a change in the state representation after the at least one of the one or more medical images is assigned to one of the plurality of radiologists, and
adjust the model based on the feedback.

12. The system of claim 11, wherein metadata of each of the one or more medical images includes at least one of an arrival time of the medical image, a due time of the medical image, a modality of the medical image, a procedure of the medial image, a body part of the medical image, and a description of the medical image.

13. The system of claim 11, wherein the radiologist information includes at least one of a specialty of each of the plurality of radiologists, a work list of each of the plurality of radiologists, an availability of each of the plurality of radiologists, a preference of each of the plurality of radiologists, and a processing rate of each of the plurality of radiologists.

14. The system of claim 11, wherein the state representation is a 2D table comprising:
a first row including the medical image metadata, and
subsequent rows including the radiologist information, wherein each row includes metadata regarding an individual radiologist.

15. The system of claim 11, the feedback includes a rejection of the assigned one or more medical images.

16. The system of claim 11, wherein the electronic processor is further configured to provide a fairness-criteria to the model, the fairness-criteria including a plurality of conditions associated with the assignment of the one or more medical images.

17. The system of claim 16, wherein the electronic processor is further configured to calculate the feedback based on the fairness-criteria.

18. Non-transitory computer-readable medium storing instructions that, when executed by an electronic processor, perform a set of functions, the set of functions comprising:
receiving one or more medical images included in a medical study, each of the one or more medical images including image metadata defining characteristics of the corresponding medical image,
receiving radiologist metadata for each one of the plurality of radiologists,
generating a state representation of the image metadata and the radiologist metadata,
providing the state representation to the model,
assigning, with the model, at least one of the one or more medical images to one of the plurality of radiologists,
calculating feedback based on a change in the state representation after the at least one of the one or more medical images is assigned to one of the plurality of radiologists, and
adjusting the model based on the feedback.

19. The non-transitory computer-readable medium of claim 18, wherein metadata of each of the one or more medical images includes at least one of an arrival time of the medical image, a due time of the medical image, a modality of the medical image, a procedure of the medial image, a body part of the medical image, and a description of the medical image.

20. The non-transitory computer-readable medium of claim 18, wherein the radiologist information includes at least one of a specialty of each of the plurality of radiologists, a work list of each of the plurality of radiologists, an availability of each of the plurality of radiologists, a preference of each of the plurality of radiologists, and a processing rate of each of the plurality of radiologists.
